(19) United States
(12) Patent Application Publication
(10) Pub. No.: US 2026/0348777 A1
(43) Pub. Date: Nov. 5, 2026

(54) SYSTEM AND METHOD FOR FORCE SENSING USING ULTRASOUND WAVEGUIDE

(71) Applicant: CALIFORNIA INSTITUTE OF TECHNOLOGY, Pasadena, CA (US)

(72) Inventors: Alexandros ROSAKIS, Altadena, CA (US); Alessio TAMBORINI, Pasadena, CA (US); Morteza GHARIB, Altadena, CA (US)

(21) Appl. No.: 19/666,102

(22) Filed: May 1, 2026

Related U.S. Application Data
(60) Provisional application No. 63/798,402, filed on May 1, 2025.
(60) Provisional application No. 63/960,601, filed on Jan. 14, 2026.

(51) Int. Cl. G01L 1/16

(52) U.S. Cl. CPC G01L 1/16

(57) ABSTRACT

The disclosure relates to techniques for contact sensing and force measurement using ultrasound waveguides. In some implementations, a system includes an ultrasound waveguide, an ultrasound transducer assembly, and a processing device. The ultrasound transducer assembly is configured to acoustically couple to the ultrasound waveguide, generate an ultrasound wave signal that travels along a major axis of the ultrasound waveguide and produces one or more partial reflections associated with one or more contact locations of the ultrasound waveguide having an altered acoustic impedance due to externally applied contact force at the contact locations, and receive a reflected ultrasound waveform signal including data representative of the partial reflections. The processing device is configured to estimate, based on the reflected ultrasound waveform signal, each of the contact locations. The processing device can also be configured to estimate, using at least amplitude information of the reflected ultrasound waveform signal, the externally applied contact force.

### CROSS-REFERENCE TO RELATED APPLICATIONS

This application claims the benefit of U.S. Provisional Patent Application No. 63/798,402, filed May 1, 2025, and titled “System and Method for Haptic Sensing Using Ultrasound Waveguides”, and U.S. Provisional Patent Application No. 63/960,601 filed Jan. 14, 2026, and titled “System and Method for Haptic Sensing Using Ultrasound Waveguides”. All of the above applications are incorporated herein by reference in their entirety.

### BACKGROUND

The sense of touch enables humans to perceive and interact with the world by providing real-time feedback regarding texture, pressure, and contact dynamics. In robotics and human-machine interfaces, contact sensing and/or force measurement can enable reliable manipulation and interaction with real-world environments. Many existing contact-sensing technologies, however, face limitations in scalability and form factor. For example, expanding a sensing area while maintaining spatial and temporal resolution typically requires an increase in the number of sensors, which can increase system complexity, wiring density, power consumption, and/or processing demands. Additionally, many systems have limited flexibility and may be unable to conform to complex surfaces.

### SUMMARY

The technology described herein relates to systems and methods for contact sensing and/or force measurement using ultrasound waveguides, referred to herein as ultrasound force sensing (UFS).

In one embodiment, a system comprises an ultrasound waveguide; an ultrasound transducer assembly configured to acoustically couple to the ultrasound waveguide, the ultrasound transducer assembly configured to: generate an ultrasound wave signal that travels along a major axis of the ultrasound waveguide and produces one or more partial reflections associated with one or more contact locations of the ultrasound waveguide having an altered acoustic impedance due to an externally applied contact force at the one or more contact locations; receive a reflected ultrasound waveform signal including data representative of the one or more partial reflections; and a processing device configured to estimate, based on the reflected ultrasound waveform signal, each of the one or more contact locations.

In some implementations, estimating, based on the reflected ultrasound waveform signal, each of the one or more contact locations comprises determining, based on an ultrasound wave speed inside the ultrasound waveguide, and a measured time from transmission of the ultrasound wave signal to reception of the reflected ultrasound waveform signal, each of the one or more contact locations.

In some implementations, the processing device is further configured to estimate, using at least amplitude information of the reflected ultrasound waveform signal, the externally applied contact force at the one or more contact locations.

In some implementations, the one or more contact locations include a plurality of contact locations; and estimating the externally applied contact force at the one or more contact locations comprises estimating a force applied at each of the plurality of contact locations.

In some implementations, the one or more contact locations include a plurality of contact locations; and estimating the externally applied contact force at the one or more contact locations comprises estimating a continuous force profile across a region of the ultrasound waveguide that includes the plurality of contact locations.

In some implementations, estimating, using at least the amplitude information of the reflected ultrasound waveform signal, the externally applied contact force at the one or more contact locations comprises mapping, based on force versus amplitude calibration data associated with the ultrasound waveguide, the amplitude information to force information.

In some implementations, the reflected ultrasound waveform signal comprises a time-domain voltage signal; and estimating the one or more contact locations and the externally applied contact force comprises mapping the time-domain voltage signal to an output indicative of force as a function of position along the ultrasound waveguide.

In some implementations, the system further comprises a sheath encapsulating the ultrasound waveguide, the sheath configured to transmit the externally applied contact force to the ultrasound waveguide at the one or more contact locations.

In some implementations, an inner surface of the sheath comprises one or more indenters oriented toward the ultrasound waveguide, each of the one or more indenters configured to contact a respective one of the one or more contact locations responsive to the externally applied contact force.

In some implementations, each of the one or more indenters is oriented to transfer the externally applied contact force to the ultrasound waveguide along a single axis.

In some implementations, the system further comprises a component having a contact surface mechanically coupled to the ultrasound waveguide or a sheath encapsulating the ultrasound waveguide, the component configured to transfer the externally applied contact force from the contact surface to the ultrasound waveguide or the sheath, and the processing device configured to sense contact at the contact surface by estimating, based on the reflected ultrasound waveform signal, each of the one or more contact locations.

In some implementations, the component is a gripping device configured to generate the externally applied contact force at the contact surface.

In some implementations, the component is a medical device or a robotic device.

In some implementations, prior to estimating each of the one or more contact locations, the processing device is further configured to remove, using a reference signal obtained when no external contact force is applied to the ultrasound waveguide, noise from the reflected ultrasound waveform signal.

In some implementations, the ultrasound waveguide has a circular or rectangular cross section.

In some implementations, the ultrasound waveguide is a metallic sheet waveguide having a rectangular cross section.

In some implementations, the ultrasound waveguide is a wire waveguide having a circular cross section.

In some implementations, the ultrasound transducer assembly comprises a piezoelectric transducer.

In some implementations, the ultrasound transducer assembly comprises a magnetostrictive transducer including a first coil of wire configured to be placed around a first portion of the wire waveguide, and the magnetostrictive transducer is configured to generate the ultrasound wave signal by passing a time-varying current through the first coil of wire placed around the first portion of the wire waveguide in the presence of a bias magnetic field.

In some implementations, the magnetostrictive transducer is configured to receive the reflected ultrasound waveform signal via the first coil of wire.

In some implementations, the magnetostrictive transducer further comprises a second coil of wire configured to be placed around a second portion of the wire waveguide, and the magnetostrictive transducer is configured to receive the reflected ultrasound waveform signal via the second coil of wire.

In one embodiment, a method comprises generating, by an ultrasound transducer assembly acoustically coupled to an ultrasound waveguide, an ultrasound wave signal that travels along a major axis of the ultrasound waveguide and produces one or more partial reflections associated with one or more contact locations of the ultrasound waveguide having an altered acoustic impedance due to an externally applied contact force at the one or more contact locations; receiving, by the ultrasound transducer assembly, a reflected ultrasound waveform signal including data representative of the one or more partial reflections; and estimating, by a processing device, based on the reflected ultrasound waveform signal, each of the one or more contact locations.

In some implementations, the method further comprises applying the externally applied contact force at the one or more contact locations.

In some implementations, applying the externally applied contact force comprises applying the externally applied contact force via a contact surface of a device coupled to the ultrasound waveguide.

In some implementations, the device is a gripping device, the contact surface is a gripping surface of the gripping device, and applying the externally applied contact force comprises gripping an object with the gripping device.

In some implementations, the method further comprises estimating, by the processing device, using at least amplitude information of the reflected ultrasound waveform signal, the externally applied contact force at the one or more contact locations.

In some implementations, the reflected ultrasound waveform signal comprises a time-domain voltage signal; and estimating the one or more contact locations and the externally applied contact force comprises mapping the time-domain voltage signal to an output indicative of force as a function of position along the ultrasound waveguide.

As used herein, the term “acoustically coupled” refers to an arrangement that enables transmission of ultrasonic energy between components, including via mechanical contact and/or via non-contact coupling mechanisms. For example, in magnetostrictive implementations, an excitation coil can be magnetically coupled to a waveguide (e.g., via a time-varying magnetic field and a magnetic bias field) to induce magnetostrictive strain that generates ultrasonic waves in the waveguide. In addition, the strain of magnetized particles can generate a time varying magnetic field that induces a measurable voltage in a receiver coil that is magnetically coupled to the waveguide. Acoustic coupling can be permanent or removable.

Other features and aspects of the disclosed technology will become apparent from the following detailed description, taken in conjunction with the accompanying drawings, which illustrate, by way of example, the features in accordance with implementations of the disclosed technology. The summary is not intended to limit the scope of any inventions described herein, which are defined by the claims and equivalents.

### BRIEF DESCRIPTION OF THE DRAWINGS

The present disclosure, in accordance with one or more implementations, is described in detail with reference to the following figures. The figures are provided for purposes of illustration only and merely depict example implementations. Furthermore, it should be noted that for clarity and ease of illustration, the elements in the figures have not necessarily been drawn to scale.

FIG. 1 depicts an example system, including operation of an ultrasound waveguide, within which the technology described herein can be implemented to provide UFS, in accordance with some implementations of the disclosure.

FIG. 2 depicts different shapes of waveguides that can be used to perform UFS in accordance with some implementations of the disclosure.

FIG. 3A depicts an example implementation of a waveguide that is encapsulated in a sheath configured to transmit an externally applied contact force to the ultrasound waveguide at one or more contact locations, in accordance with some implementations of the disclosure.

FIG. 3B depicts an example implementation of a waveguide that is encapsulated in a sheath have an inner surface with indenters, in accordance with some implementations of the disclosure.

FIG. 4 shows a time-domain voltage signal when an ultrasound waveguide is not subject to any external forces, and a time-domain voltage signal when the ultrasound waveguide is subject to one external force, in accordance with some implementations of the disclosure.

FIG. 5 shows an ultrasound waveguide that is subject to a sliding or moving external force, and a corresponding time-domain voltage signal, in accordance with some implementations of the disclosure.

FIG. 6 shows an ultrasound waveguide that is subject to multiple external forces, and a corresponding time-domain voltage signal, in accordance with some implementations of the disclosure.

FIG. 7 illustrates three example force versus signal reflection amplitude calibration curves derived for different materials on a steel waveguide, in accordance with some implementations of the disclosure.

FIG. 8 is an operational flow diagram illustrating an example method that can be implemented to estimate one or more external contact locations and an associated force at an ultrasound waveguide using UFS, in accordance with some implementations of the disclosure.

FIG. 9 is an operational flow diagram illustrating an example method that can be implemented to automate the operation of a device based on force sensed at a contact surface of the device using an ultrasound waveguide, in accordance with some implementations of the disclosure.

FIG. 10 depicts an example application of a single wire waveguide that is acoustically coupled to an ultrasound transducer, and that is incorporated in a robotic hand manipulator, in accordance with some implementations of the disclosure.

FIG. 11 conceptually illustrates one example implementation of an UFS system having a piezoelectric transducer that can be removably and acoustically coupled to a waveguide, in accordance with some implementations of the disclosure.

FIG. 12A illustrates one example implementation of an UFS system having a magnetostrictive transducer with a transduction coil of wire, in accordance with some implementations of the disclosure.

FIG. 12B conceptually illustrates the magnetostrictive transducer of FIG. 12A being removably coupled to the wire waveguide of the UFS system of FIG. 12A, in accordance with some implementations of the disclosure.

FIG. 12C illustrates on example implementation of a UFS system having a magnetostrictive transducer that includes an excitation coil and a receiver coil, in accordance with some implementations of the disclosure.

FIG. 12D illustrates one example implementation of a wire waveguide that includes a portion with a coating that increases the magnetostrictive coefficient of the portion, in accordance with some implementations of the disclosure.

FIG. 12E illustrates an example implementation of a UFS system that includes multiple transduction coils coupled to a wire waveguide, in accordance with some implementations of the disclosure.

The figures are not exhaustive and do not limit the present disclosure to the precise form disclosed.

### DETAILED DESCRIPTION

The technology described herein relates to systems and methods for contact sensing and/or force measurement using ultrasound waveguides, referred to herein as UFS. In accordance with implementations of the disclosure, an ultrasound transmitter is coupled to a waveguide such that the waveguide acts as an ultrasound waveguide. Ultrasound waves transmitted by the ultrasound transmitter travel along a major axis of the waveguide until they reach an area with a different acoustic impedance in the waveguide caused by a contact force between the waveguide and an external material. Such an area can be referred to as a contact location. The waves reflect from one or more such contact locations in proportion to the change in acoustic impedance, with larger changes creating larger reflections. The reflected ultrasound waveform is collected using an ultrasound receiver/sensor.

The UFS systems and methods described herein leverage the fact that the received waveform encodes location and force information. Some implementations of the disclosure relate to performing time of flight measurements between transmission and reception of ultrasound waves via the ultrasound waveguide to determine one or more contact locations on the ultrasound waveguide. Further implementations of the disclosure relate to estimating, based on amplitude information of the received waveform, one or more forces along the one or more contact locations. In accordance with such implementations, force versus amplitude calibration data associated with the ultrasound waveguide can be determined in advance of operation.

Further implementations of the disclosure are directed to designing the UFS system to modulate the received waveform in a predictable manner. In accordance with such implementations, the bare waveguide can be encapsulated with a sheath of known material that presses into the waveguide when force is applied to the external sheath. The material properties of the sheath and the waveguide can be tuned to adjust the sensitivity and range of the UFS systems and methods described herein.

Various advantages can be realized by implementing the technology described herein. By virtue of using an ultrasound waveguide to perform contact sensing and/or force measurements, the technology described herein provides a scalable system that does not necessarily require an increasing number of sensors to perform contact sensing and/or force measurements over a larger sensing area. In some implementations, a single transducer and waveguide can be used to perform measurements. In addition, by virtue of performing UFS using a waveguide having an adaptable formfactor (e.g., wire, sheet, etc.) and material properties, the technology described herein can be readily adapted to any application, offering a flexible approach to contact sensing. As such, the UFS technology described herein can bring scalability advantages over current technologies, providing a highly applicable solution for contact and force sensing into a potentially wide range of applications such as robotic appendages, motion capture suits, tactile controls, and the like. Further still, by virtue of tuning the material properties of the waveguide and/or a sheath that contacts the waveguide during contact sensing, UFS can be performed in a predictable manner with a known sensitivity and/or range of sensing. These and other advantages that can be realized by implementing the technology described herein are further exemplified by the description that follows.

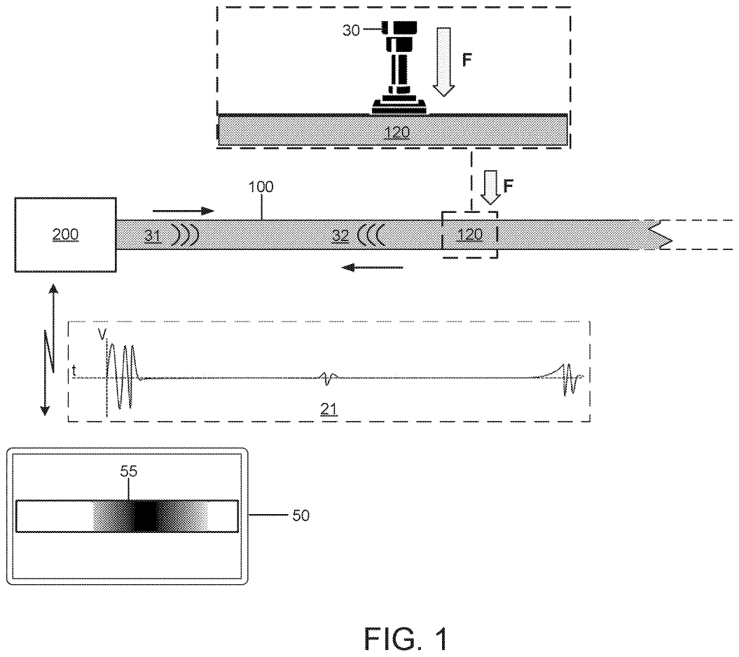

FIG. 1 depicts an example system, including operation of a waveguide 100, within which the technology described herein can be implemented to provide UFS, in accordance with some implementations of the disclosure. As depicted, the system includes an ultrasound transducer assembly 200 acoustically coupled to a waveguide 100 such that it functions as an ultrasound waveguide. The system also includes a force measurement device 50 in communication with ultrasound transducer assembly 200.

During operation, waveguide 100 can have one or more contact locations 120 along its major axis that are subject to an external contact force (F). The example of FIG. 1 depicts one such example contact location 120 subject to a contact force transferred from a contact surface of component 30 that is coupled to the waveguide. Component 30 can be a specific device (e.g., robotic arm) or object (e.g., shoe) that directly or indirectly couples to the waveguide surface to transmit an external force. In some implementations, the waveguide 100 can be incorporated in component 30. The contact force at each contact location 120 changes the acoustic impedance of ultrasound waveguide 100 and creates an ultrasound reflection site in the waveguide.

In operation, an ultrasound transducer of ultrasound transducer assembly 200 can be driven by a signal generator to generate an ultrasound wave signal 31 (e.g., pulse) that travels along the major axis or length of waveguide 100 and partially reflects from each contact location 120. The ultrasound wave can reflect from each contact location 120 in proportion to the change in acoustic impedance. The amplitude of the resulting ultrasound reflections can depend on the contact force between the waveguide 100 and the external material. As such, the reflected ultrasound waves/pulses 32 can encode position and force information of each contact location 120 that is carried back to the ultrasound transducer assembly 200 and recorded. This position and force information can be encoded in the form of time-of-flight and amplitude signatures, respectively.

As depicted in this example, the reflected waveform signal that is recorded comprises a time-domain voltage signal 21. Recorded signals can be analyzed by force measurement device 50 to compute the contact locations and/or external forces along the waveguide 100, and/or to perform related functions.

Depending on the application, various waveguides having different material properties and/or shapes could be used for contact sensing. The waveguide 100 can be made of any suitably elastic material that returns to shape after being subject to an external force. For example, the waveguide 100 can be formed of metals or metal alloys such as stainless steel, titanium, nickel-titanium, and the like. The waveguide 100 could also be formed of non-metallic materials such as polymers or ceramics, particularly those that are flexible and suitable for use as ultrasound waveguides. In implementations where waveguide 100 is used with a magnetostrictive transducer, waveguide 100 can be made of or contain a material that has a higher magnetostrictive coefficient than some of the foregoing materials, thereby creating a stronger ultrasound signal in the waveguide. For example, a waveguide 100 made of stainless steel can contain areas that are coated with nickel. As further described below, in such implementations one or more portions of waveguide 100 can be permanently magnetized or magnetized during use.

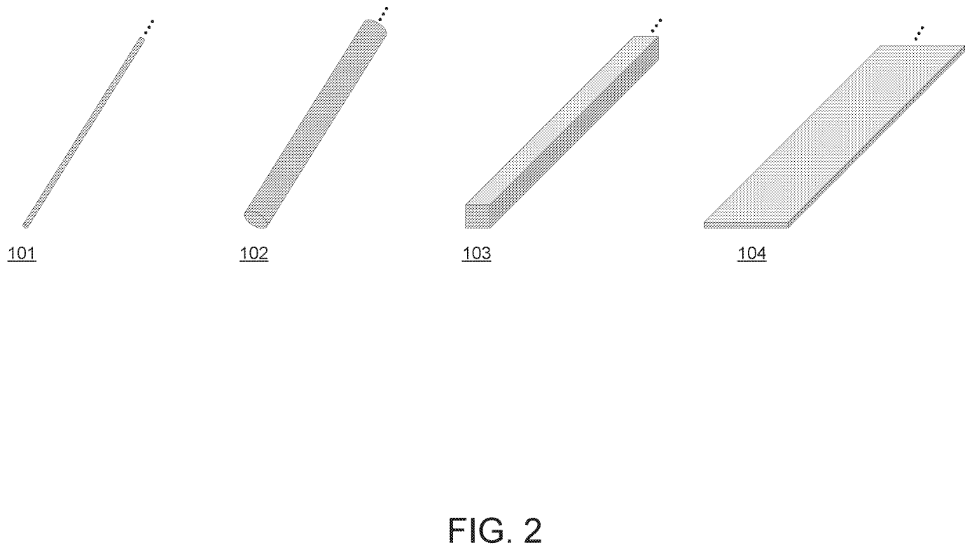

The waveguide 100 can have a circular cross section, a rectangular cross section, or some other cross section that is suitable for the UFS application. FIG. 2 depicts different shapes of waveguides that can be used to perform UFS in accordance with implementations of the disclosure. Depending on the UFS application, a suitable waveguide shape can be adapted for the application. Waveguide 101 is illustrated as a wire waveguide. Example UFS applications that a wire waveguide 101 can be suitable for include medical device applications such as contact and/or force sensing with a catheter or endoscope. Waveguide 102 is illustrated as a rod waveguide. Example UFS applications that a rod waveguide 102 can be suitable for include building struts or control interfaces such as a joystick. Waveguide 103 is illustrated as a rectangular waveguide. An example UFS application that a rectangular waveguide 103 can be suitable for includes a multicomponent load cell. Waveguide 104 is illustrated as a planar or sheet waveguide. Example UFS applications that a sheet waveguide 104 can be suitable for include a control sensitive skin for robots or bottom soles of feet for humans, robots, or humanoids.

In some implementations where waveguide 100 is a wire, the diameter of the wire can be anywhere between about 0.1 mm and 150 mm. In some implementations where waveguide 100 is a sheet (e.g., a thin metal sheet), the thickness of the metal sheet can be anywhere between about 0.1 mm and 100 mm. The area of the sheet can be sized as needed to provide coverage over the sensing area. It should be appreciated that the material and diameter/thickness of the waveguide wire or waveguide sheet can be specifically adapted to a UFS application.

The acoustic impedance of waveguide 100 can be altered at specific locations by putting a material with a similar acoustic impedance in contact with the waveguide. For example, as depicted by FIG. 1, this could be achieved by a single bare waveguide 100 (e.g., a metal waveguide) that is directly contacted by a contact surface of component 30 having a similar acoustic impedance. In that case, contact forces on the surface create reflection sites depending on the material properties of the contacting object. In this implementation, the bare waveguide 100 could be capable of contact and force sensing and be sensitive to high acoustic impedance materials such as metals and glasses.

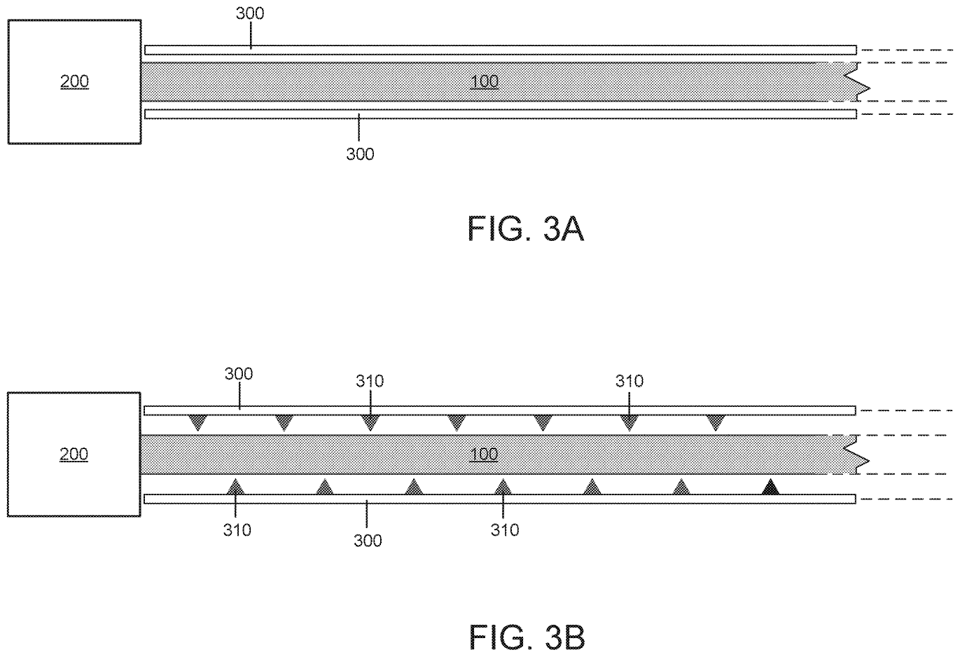

Alternatively, the waveguide 100 can be encapsulated in a sheath, either fully or partially, to modulate the effect that contact forces have on it. FIG. 3A depicts one such example implementation of a waveguide 100 that is encapsulated in a sheath 300, the sheath 300 configured to transmit an externally applied contact force to the ultrasound waveguide at one or more contact locations. By virtue of using a sheath 300 with known material properties to transmit the externally applied contact force, the reflected waveform signal can be modulated in a predictable and desired manner. The sheathed waveguide could be capable of contact and force sensing that would be sensitive to a wider range of materials of the contact surface of component 30. The outer material of sheath 300 could effectively insulate the waveguide 100 from external forces (e.g., it could be made of a soft material with a low wave speed) and have harder parts attached to its inner surface.

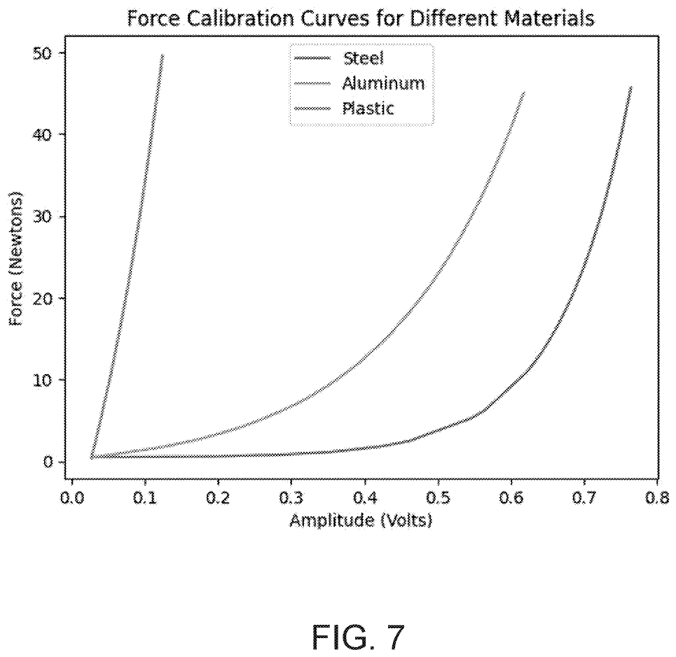

The insulating or outer surface of sheath 300 can be formed of soft materials such as a rubber or silicone. The inner surface of sheath 300 that contacts the surface of waveguide 100 could be formed of metallic or non-metallic materials. For example, for low sensitivity sensing applications, the inner surface can be formed of plastic or PLA. For high sensitivity applications, the inner surface can be formed of metals such as steel, titanium, etc. Depending on the design of external sheath 300, the same waveguide 100 can be designed to have different calibration factors, particularly a reflected waveform amplitude to force mapping as further described below with reference to FIG. 7.

In some implementations, an inner surface of the sheath can include one or more indenters oriented toward the waveguide 100, each indenter configured to contact a respective contact location responsive to an externally applied contact force. FIG. 3B depicts one such example implementation of a waveguide 100 that is encapsulated in a sheath 300 have an inner surface with indenters 310. In this implementation, each indenter 310 would create a reflection in a contact location of the waveguide 100 when the outer sheath is pressed in that area. In some implementations, the location of indenters 310 can be controlled to measure forces acting on the waveguide 100 in specific directions. For example, indenters 310 can be arranged to measure forces acting on the waveguide 100 along only one axis. Alternatively, indenters can be arranged to measure forces acting on the waveguide 100 along multiple axes. The inner surface of sheath 300 can be arranged with indenters 310 in a suitable pattern to achieve UFS in a specific direction or multiple directions. For example, in the case of single direction force measurement, the indenters 310 can be distributed along the inner surface of sheath 300 such that they contact the waveguide 100 substantially orthogonally along one axis to a produce a measurable signal. When this example is applied to a rectangular waveguide 103, the indenters can be arranged to contact only one surface of the rectangular waveguide 103.

The ultrasound transducer assembly 200 can be implemented as any assembly with an ultrasound transducer that can be acoustically coupled to waveguide 100, excite ultrasound waves along a major axis of waveguide 100, and measure the reflected ultrasound waves. The ultrasound transducer assembly 200 can include transmit and/or receive circuitry and an ultrasound transducer that can be operatively coupled to a signal generator. The signal generator can be configured to generate one or more excitation signals (e.g., pulses, tone bursts, or other waveforms). When present, the transmit circuitry can be configured to condition and/or deliver the excitation signals to the transducer of the ultrasound transducer assembly 200 such that the transducer emits corresponding ultrasonic waves. When present, the receive circuitry can be configured to receive and amplify electrical signals generated by the transducer in response to reflected ultrasound waves, the electrical signals being representative of the received ultrasonic waves.

In some implementations, the ultrasound transducer can be a piezoelectric transducer such as a piezoelectric disk. For example, the piezoelectric disk can be used in conjunction with a pulser-receiver, where the pulser section can be configured to generate electrical excitation pulses that are converted into ultrasonic pulses by the piezoelectric transducer when applied thereto, and the receiver section is configured to amplify voltage signals produced by the piezoelectric transducer that are representative of received ultrasonic pulses. In other implementations, the ultrasound transducer can be a magnetostrictive transducer that is magnetically coupled to a magnetized waveguide (e.g., a magnetized wire). The magnetostrictive transducer can include one or more removable coils of wire that are placed around a magnetized waveguide to induce and/or measure propagating ultrasound waves. When a time-varying current is passed through an excitation or transduction coil, the coil can generate a time-varying magnetic field that produces magnetostrictive strain in the wire waveguide, thereby generating ultrasound waves in the wire waveguide that propagate away from the coil.

In yet other implementations, the ultrasound transducer can use any other suitable transducer technology for generating and detecting ultrasound waves in waveguide 100.

As depicted in the example of FIG. 1, a single ultrasound transducer of ultrasound transducer assembly 200 can be acoustically coupled to an end of waveguide 100 to create an ultrasound waveguide and perform UFS using the reflected ultrasound waveform collected at the ultrasound transducer device. In this configuration, the UFS system can be simplified in cost and overall complexity. For example, in cases where the waveguide 100 is a wire waveguide, the wire waveguide can be contact and force sensitive along the entire length of the wire. The location of contact sensing on the wire can be identified by measuring the time it takes from the initial ultrasound pulse being generated to the return of the reflected wave packets. The sensing area of the wire can simply be increased by increasing the length of the wire waveguide. As such, a single ultrasonic transducer can measure along the entire length of the wire waveguide over several different distance scales in this example.

In alternative implementations, multiple ultrasound transducers can be acoustically coupled to waveguide 100 to perform UFS. For example, a respective ultrasound sensor could be positioned to collect a reflected ultrasound signal generated from a respective contact location 120 or multiple respective contact locations 120. Such implementations may reduce the difficulty in extracting an ultrasound signal reflected from a respective contact location 120 as the signal would no longer be convolved with other reflected signals or convolved with fewer reflected signals, at the cost of a more complex and costly hardware arrangement. As further described below, a magnetostrictive transducer may obviate the complexity of such a hardware arrangement.

In implementations where UFS is performed along multiple dimensions of the waveguide 100 (e.g., along a major axis and a minor axis), multiple ultrasound emitters can be acoustically coupled to waveguide 100.

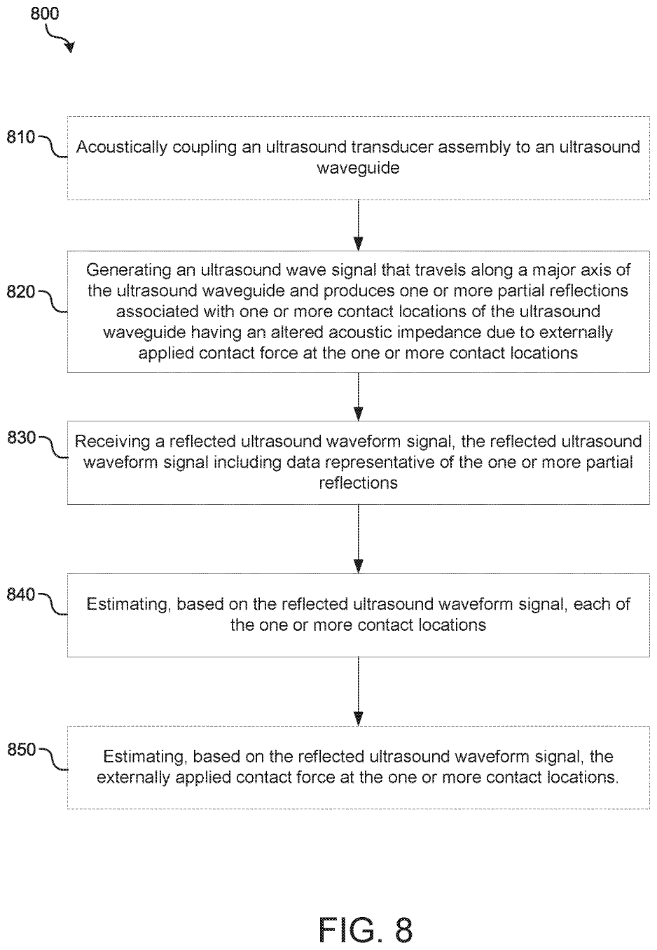

Force measurement device 50 is configured to compute the contact locations and/or external forces along the waveguide 100 based on the reflected ultrasound waveform signal data (e.g., time-domain voltage signal 21) collected by ultrasound transducer assembly 200. To this end, force measurement device 50 can be configured to decode the location and/or force information encoded within the signal based on analytical models, machine learning solutions, and/or a combination thereof. To perform these operations, measurement device 50 can extract time of flight and amplitude information from the received signal. Particular techniques for decoding such information are further described below with reference to FIG. 8.

The reflected ultrasound waveform data can be communicated from ultrasound transducer assembly 200 to force measurement device 50 via a wired or a wireless communication link. For example, a radio frequency link such as a Bluetooth® link or a Wi-Fi® link can be used to communicate the data.

In this particular example, force measurement device 50 is illustrated as a mobile device in communication with ultrasound transducer assembly 200. For example, the mobile device can be a tablet, laptop, a smartphone, a head mounted display (HMD), or other suitable mobile device configured to estimate the contact locations and/or forces. In other implementations, force measurement device 50 can be a desktop computer or any other device configured with a processing device that can estimate the contact locations and/or forces. In yet other implementations, force measurement device 50 and ultrasound transducer assembly 200 can be integrated as part of the same assembly that performs the hardware and software functions of both devices. In a particular implementation, the force measurement device 50 can run an application for performing UFS. As depicted in this example, the application can be configured to display a numerical and/or graphic representation of the contact location and/or force information encoded in the reflected ultrasound waveform data received from ultrasound transducer assembly 200. For example, in FIG. 1 the display outputs a heatmap 55 that represents the force(s) and contact location(s) 150 as a continuous force profile across a major axis of the waveguide 100. The display can be provided in real-time to an operator of a UFS system.

In other implementations, the calculated contact locations and/or forces can be provided as inputs to other components of the system to perform other functions. In such implementations, it may not be necessary to generate a display as described. For example, such inputs may be provided to a robotic system or other system that automates additional functions based on the calculated contact locations and/or forces.

Many different applications can be realized using the UFS techniques described herein. For example, in some example applications, a gripping device such as a handheld tool or a robotic gripper can be modified with a force sensing waveguide on a grasping surface of the gripping device to identify when an object is being grasped, and the force with which the object is being grasped. For instance, in one example experiment it was observed by the inventors that a sensing system was able to determine i) when forceps modified with a force sensing waveguide gripped a cardboard shim at their tip with minimal force, and ii) when the forceps fully gripped the carboard shim, measuring more force along a larger length of the sensing area.

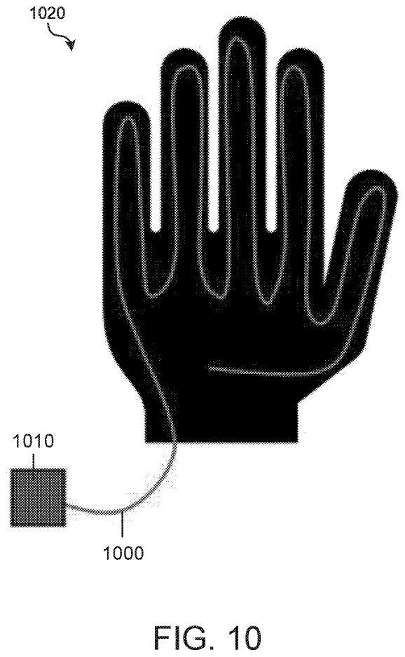

FIG. 10 depicts an example application of a single wire waveguide 1000 that is acoustically coupled to an ultrasound transducer 1010, and that is incorporated in a robotic hand manipulator 1020. The illustrated example sensorizes the entire hand, including each finger and the palm of the robotic hand manipulator 1020. The example of FIG. 10 can be accomplished using a single electrical connection, and the wire waveguide 1000 can provide the robot with touch and grip information.

Other example applications include incorporation of a sheet waveguide 104 in a surface of an object such as a robot to provide force sensing feedback over a two-dimensional surface. Yet further example applications include incorporation of a waveguide in a medical device such as an endoscope or catheter to provide force-sensing feedback. For example, the waveguide can be used to detect contact at a tip of an endoscope or catheter.

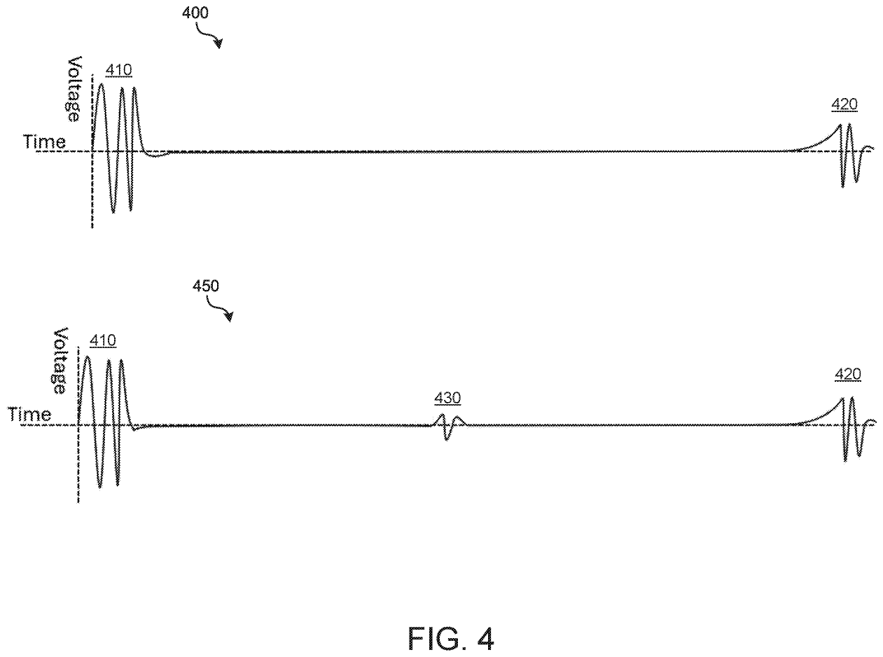

To appreciate some of the techniques described herein for decoding the location and/or force information encoded in a reflected ultrasound waveform signal data (e.g., time-domain voltage signal 21), it is instructive to consider how such a waveform signal can respond to external forces at different contact locations. To this end, FIGS. 4-6 conceptually illustrate various time-domain voltage signals and an associated configuration of a waveguide 100 configured to perform UFS, in accordance with some implementations of the disclosure.

FIG. 4 shows a time-domain voltage signal 400 when the waveguide 100 is not subject to any external forces, and a time-domain voltage signal 450 when the waveguide 100 is subject to one external force. In time-domain voltage signal 400, the ultrasound transducer assembly 200 can measure an excitation pulse 410 used to create the ultrasound wave. After a delay, the reflection/echoes 420 from the end of the waveguide 100 are recorded. Time-domain voltage signal 450 records a material pressing into the waveguide 100 about halfway along its length. The transducer measures the reflection/echoes 430 created by the interaction with the contact force earlier than the reflection 420 that returns from the end of waveguide 100. The time between transmitting the excitation pulse 410 and receiving the reflection 430 can be used to estimate the position of the contact force along the length of the waveguide 100.

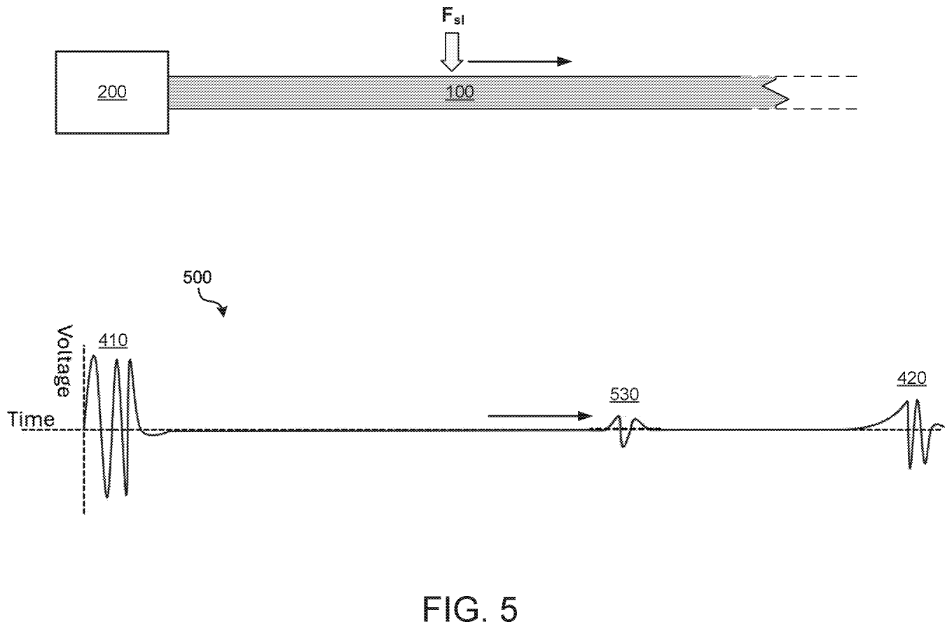

FIG. 5 shows a waveguide 100 that is subject to a sliding or moving external force (Fₛ₁), and a corresponding time-domain voltage signal 500. In this example, after the material was originally pressing into the waveguide 100 about halfway along its length (e.g., as depicted by time-domain voltage signal 450), the contact point moves further down the length of the waveguide 100. The recorded reflection/echo 530 associated with this movement takes longer to return to the transducer assembly 200. This illustrates how physical distance is encoded into the temporal domain.

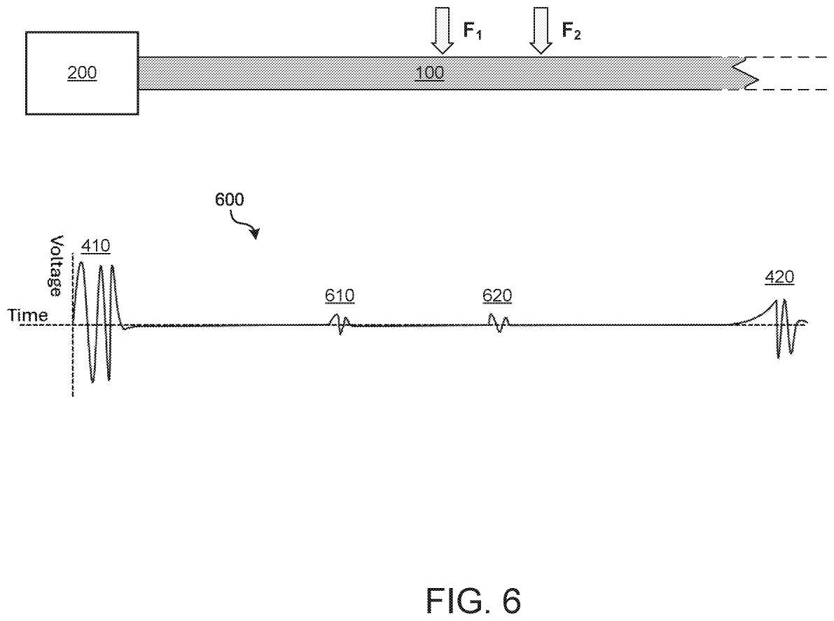

FIG. 6 shows a waveguide 100 that is subject to multiple external forces (F₁ and F₂), and a corresponding time-domain voltage signal 600. In this example, two materials (or the same material) are pressing into the waveguide 100 at different contact locations or points along its length. Each contact force creates a respective reflection/echo (610 and 620) that is encoded in signal 600 measured via transducer assembly 200. The echoes 610 and 620 can be differentiated by time-of-flight measurements to identify their position along the waveguide 100.

It should be appreciated that the foregoing examples are for the purpose of illustration only, and they may not account for factors such as system noise (e.g., at a receiver of the transducer assembly 200) and/or signal convolution from multiple signal reflections. It should also be appreciated that although the foregoing figures are described with respect to a waveguide-transducer system with a bare waveguide 100, the technology could likewise be implemented with a waveguide 100 encapsulated with a sheath 300 as described above.

To decode the force information encoded within reflected ultrasound waveform signal data, some implementations of the disclosure are directed to leveraging calibration data that maps a measured amplitude of the waveform to a contact force. As such a mapping can depend on the material properties of the waveguide 100 and the surface that directly contacts waveguide 100 (e.g., inner surface of sheath 300 or surface of component 30), the calibration data can be derived in advance of operating the waveguide 100 within an UFS system such as the system depicted in FIG. 1.

To this end, FIG. 7 illustrates three example force versus signal reflection amplitude calibration curves derived for different materials on a steel waveguide, in accordance with some implementations of the disclosure. In this example, the calibration curve maps a measured amplitude in Volts to a force in Newtons. In this example, measurements were performed by fixing a bottom indenter of a specific material (steel, aluminum, or PLA plastic) to a calibrated load cell. Then the wire waveguide was placed on top of the load cell. Finally, an additional indenter, comprised of the same material as the bottom indenter, was pressed into the waveguide. The force was measured at 10 samples per second. Each force sample triggered a pulse generator that sent waves down the length of the waveguide. The force sample also triggered a receiver circuit that measured the returning echoes. The amplitude of the echoes corresponding to the material being pressed into the waveguide was thus measured. In some implementations, the reflection amplitude can be normalized by dividing by the end reflection amplitude when the waveguide is not being contacted by anything else. The end reflection can act as an almost perfect reflector and is a measure of the total energy inside the waveguide.

FIG. 8 is an operational flow diagram illustrating an example method 800 that can be implemented to estimate one or more external contact locations and an associated force at an ultrasound waveguide 100 using UFS, in accordance with some implementations of the disclosure. The UFS systems depicted throughout the disclosure (e.g., in FIG. 1) can be used to perform method 800. In some implementations, operations 840-850 of method 800 can be performed by a force measurement device 50 in response to executing, using a processing device, instructions stored in a computer readable medium.

Optional operation 810 includes acoustically coupling an ultrasound transducer to a waveguide. The transducer can be acoustically coupled to a first end of the waveguide (e.g., wire) such that it can excite ultrasound waves along a major axis in the waveguide and measure ultrasound waves reflected from one or more contact locations of the waveguide. The one or more contact locations of the ultrasound waveguide can have an altered acoustic impedance due to externally applied contact force at the one or more contact locations.

In some implementations, acoustic coupling can occur by forming a fixed physical connection between the transducer and waveguide (e.g., via soldering) or a removable physical connection (e.g., removably coupling an end of the waveguide to the transducer). In some implementations, acoustic coupling can occur without a direct physical connection between the components. For example, a magnetostrictive transducer can be magnetically coupled to the waveguide. In some implementations, the UFS system may be preconfigured such that an ultrasound transducer is already acoustically coupled to a waveguide. In such implementations, operation 810 can be skipped.

Operation 820 includes generating an ultrasound wave signal that travels along the major axis of the ultrasound waveguide and produces one or more partial reflections associated with one the one or more contact locations having an altered acoustic impedance.

Operation 830 includes receiving a reflected ultrasound waveform signal, the reflected ultrasound waveform signal including data representative of the one or more partial reflections. In some implementations, the reflected ultrasound waveform can be saved as a voltage versus time series of datapoints.

In some implementations, the signal-to-noise ratio (SNR) of the reflected ultrasound waveform signal can be increased by obtaining an unperturbed waveguide signal (i.e., without any external force applied to the waveguide) as a reference signal and using the reference signal to reduce noise in the reflected ultrasound waveform signal. For example, suitable signal subtraction techniques can be employed. The reference signal can be obtained during a calibration period, at startup, periodically, or at another suitable time. In some implementations, the signal subtraction can be performed in the time domain.

Operation 840 includes estimating, based on the reflected ultrasound waveform signal, each of the one or more contact locations. In various implementations, time-of-flight information encoded in the received waveform data can be used to derive one or more contact locations. More particularly, the one or more contact locations can be determined based on an ultrasound wave speed inside the ultrasound waveguide, and a measured time from transmission of the ultrasound wave signal to reception of the reflected ultrasound waveform signal. The time of flight can be measured from an excitation ultrasound transducer to a receiver of the transducer.

In some implementations, the ultrasound wave speed can be predetermined, measured, and/or calibrated based on parameters such as waveguide material, temperature, and/or other parameters. The time of flight can be determined by using any suitable detection technique including, for example, thresholding, peak detection, and/or correlation.

Optional operation 850 includes estimating, based on the reflected ultrasound waveform signal, the externally applied contact force at the one or more contact locations. In various implementations, amplitude information encoded in the received waveform data can be used to estimate the externally applied contact force at the one or more contact locations. For example, as described above with reference to FIG. 7, force versus amplitude calibration data associated with the ultrasound waveguide can be used to map the amplitude information of the waveform to force information.

In some implementations, a discrete force applied at each contact location can be estimated. In some implementations, a force applied across an area including a given contact location can be estimated. In some implementations, a force applied across a region of the ultrasound waveguide that includes all or multiple of the contact locations can be estimated. For example, heatmap 55 illustrates an example of a continuous force profile measured across a major axis of a waveguide 100. As such, it should be appreciated that discrete or continuous force measurements can be obtained for the contact locations.

In some implementations, deconvolution techniques can be applied to deconvolve multiple independent force contributions. Such deconvolution techniques can employ analytical methods, machine learning, or some combination thereof.

In implementations where the reflected ultrasound waveform signal includes a time-domain voltage signal, operations 840-850 can include mapping the time-domain voltage signal to an output indicative of force as a function of position along the ultrasound waveguide.

In some implementations involving UFS across multiple dimensions, method 800 can be performed across another axis (e.g., minor axis) of the waveguide.

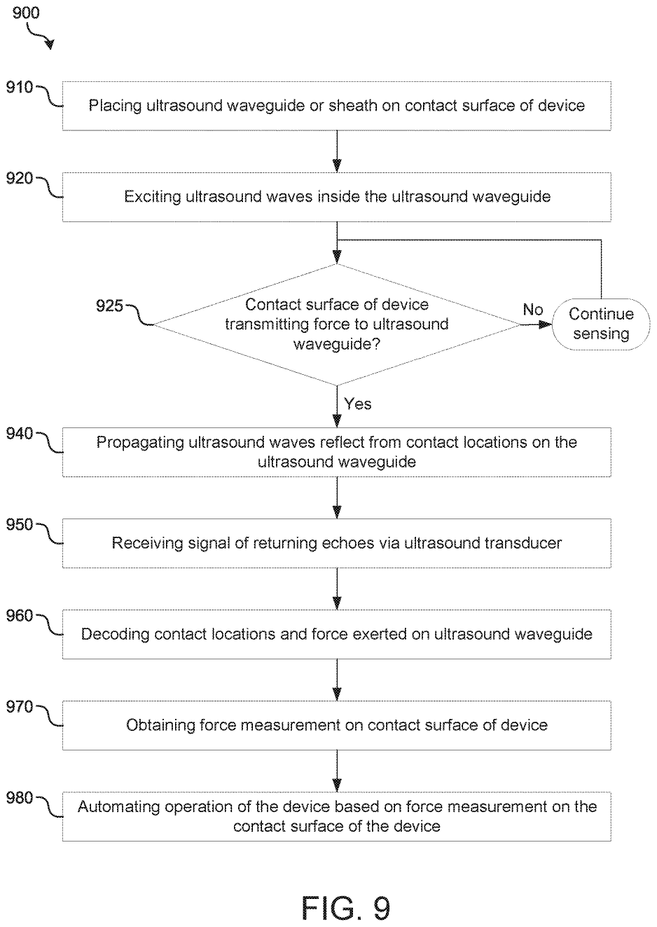

FIG. 9 is an operational flow diagram illustrating an example method 900 that can be implemented to automate the operation of a device based on force sensed at a contact surface of the device using an ultrasound waveguide, in accordance with some implementations of the disclosure. Operation 910 includes placing an ultrasound waveguide or sheath of the waveguide on a contact surface of the device. For example, if the device is a gripping device, the ultrasound waveguide can be placed on a contact surface of the gripping device for gripping. Operation 920 includes exciting ultrasound waves inside the ultrasound waveguide. Decision 925 includes determining if the contact surface of the device is transmitting force to the ultrasound waveguide. For example, if the device is a gripping device, decision 925 can also include determining if the gripping device is grabbing an object, in which case the waveguide would be pressed into the object and the gripper's contact surface. If not, sensing can continue. When no force is applied at the waveguide, ultrasound waves can propagate along a length of the waveguide without reflecting, and the ultrasound transducer may not measure returning echoes.

If yes, operation 940 includes propagating ultrasound waves reflected from the contact locations on the ultrasound waveguide. Operation 950 includes receiving a signal of returning echoes via the ultrasound transducer. The returning echoes can be generated due to a change in acoustic impedance from the transmitted force to the contact locations. Operation 960 includes decoding, from the received signal, contact locations and force exerted on ultrasound waveguide. Operation 970 includes obtaining a force measurement on a contact surface of the device. The force measurement can be obtained based on the decoded contact location(s) and force exerted on the ultrasound waveguide. For example, in the case of the gripping device, the gripper's gripping force can be measured. Operation 980 includes automating operation of the device based on the force measurement on the contact surface of the device. For example, in the case of the gripping device, operation 980 can include deciding whether the grip is secure based on the measured gripping force, and taking corrective action as needed (e.g., if the gripping force is changing, adjusting the gripping device).

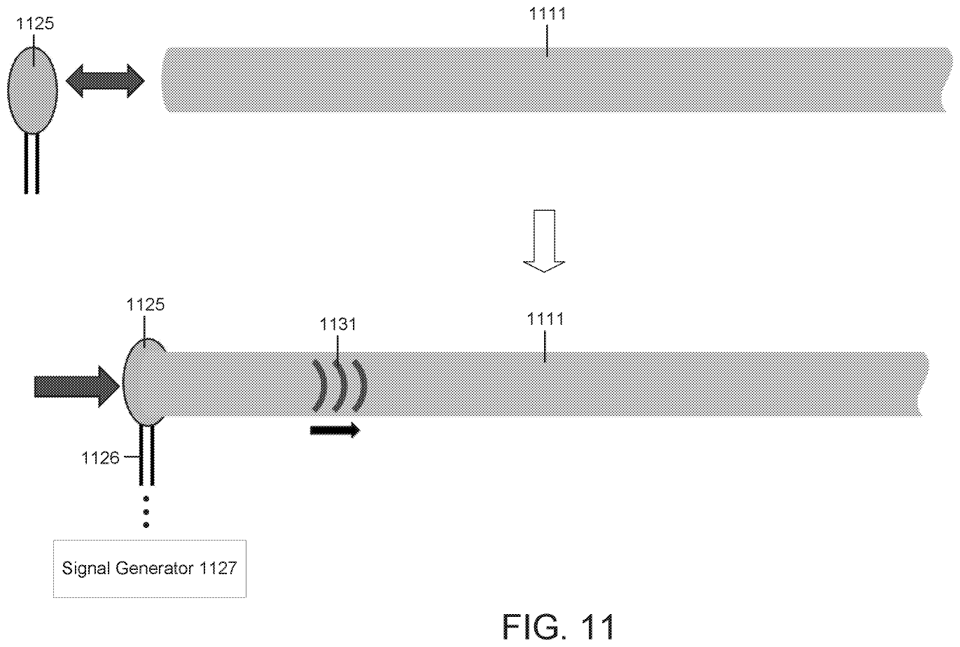

FIG. 11 conceptually illustrates one example implementation of a UFS system having a piezoelectric transducer 1125 that can be removably and acoustically coupled to a waveguide 1111, in accordance with some implementations of the disclosure. As depicted, piezoelectric transducer 1125 can be removably and acoustically coupled to the wire waveguide 1111 by application of mechanical force. Once piezoelectric transducer 1125 is mechanically coupled to the waveguide 1111, a signal generator 1127 and/or drive circuitry coupled to piezoelectric transducer 1125 via electrical connector 1126 can drive the piezoelectric transducer 1125 with an excitation signal (e.g., a pulse), thereby generating ultrasound waves 1131 that propagate along waveguide 1111. In other implementations, the piezoelectric transducer 1125 can be non-removably bonded to the waveguide 1111. For example, epoxy or some other suitable bonding mechanism can be used. In such implementations, an electrical connector 1126 that is detachable can be configured to removably couple piezoelectric transducer 1125 to signal generator 1127 and/or drive circuitry.

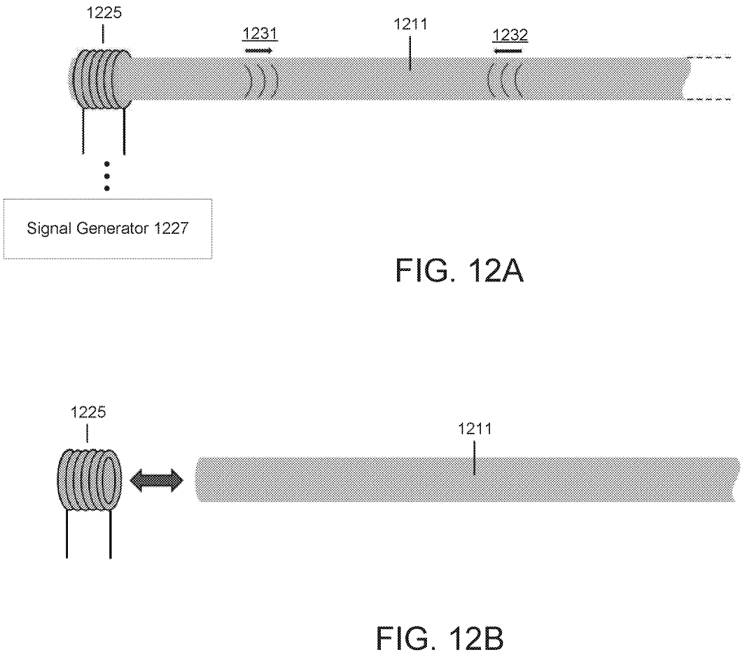

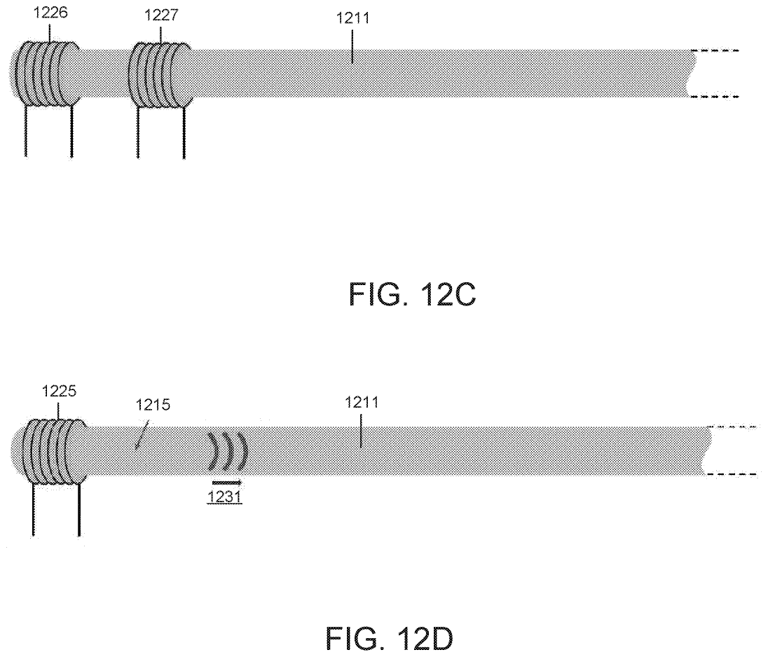

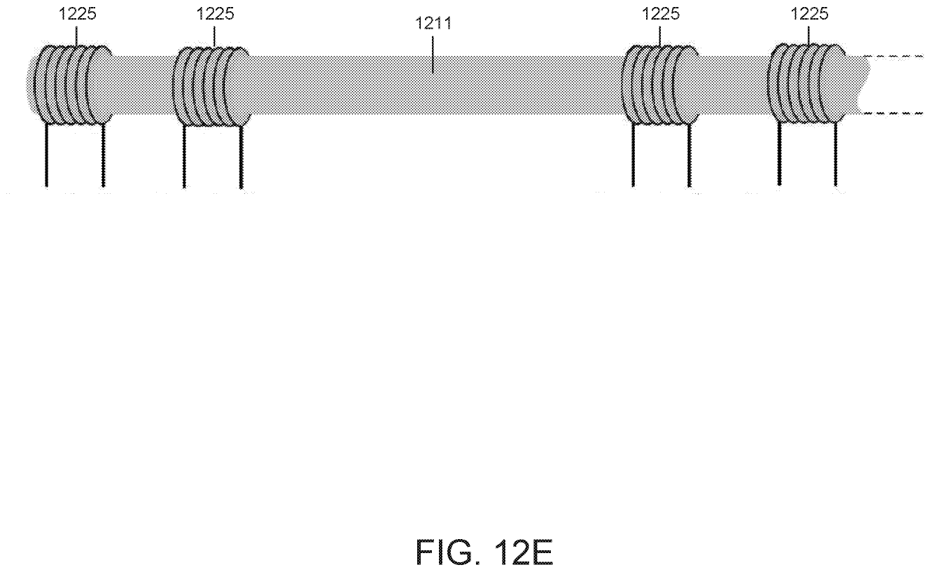

FIGS. 12A-12B conceptually illustrate one example implementation of a UFS system having a magnetostrictive transducer that can be acoustically and removably coupled to a wire waveguide 1211, in accordance with some implementations of the disclosure. As depicted, the magnetostrictive transducer can include a transduction coil of wire 1225 (“transduction coil 1225”) that can be acoustically and removably coupled to the wire waveguide 1211 by placing it around the wire waveguide 1211 as shown in FIG. 12B. In this example, the coil is acoustically coupled to the wire waveguide 1211 by magnetic coupling. In alternative implementations, the transduction coil 1225 can be configured to be fixedly attached around the wire waveguide 1211.

In operation, transduction coil 1225 is configured to use the magnetostriction effect to induce ultrasound waves 1231 and measure reflected ultrasound waves 1232 that encode contact and/or force information. To this end, wire waveguide 1211 can be magnetized prior to inducing ultrasound waves 1211. In some implementations, wire waveguide 1211 can be magnetized during operation by an external bias magnetic field. For example, a permanent magnet or an electromagnet can temporarily magnetize wire waveguide 1211 by applying an external bias magnetic field. Alternatively, at least the portion of the wire waveguide 1211 where transduction coil 1225 is placed can be permanently magnetized.

A signal generator 1227 and/or drive circuitry can be operatively coupled to the transduction coil 1225 and configured to provide one or more electrical excitation signals (e.g., pulses, tone bursts, or other waveforms) to the transduction coil 1225, thereby causing a time-varying current to flow in the transduction coil 1225 and generating a corresponding time-varying magnetic field.

When a time-varying current (e.g., an alternating current) is passed through the transduction coil 1225 placed around the magnetized portion of the wire waveguide 1211, a time-varying magnetic field is induced in the core of transduction coil 1225. The magnetic particles inside the wire waveguide 1211 are then physically moved by the time-varying magnetic field, creating ultrasound waves 1231 inside the waveguide that propagate away from the transduction coil 1225. The inverse principle (i.e., the Villari effect) can also be applied to use transduction coil 1225 to measure reflected ultrasound waves 1232 that propagate through wire waveguide 1211 and back to transduction coil 1225. In some implementations, the signal generator 1227 can include switching circuitry to facilitate transmit and receive operations using the transduction coil 1225.

As depicted in the example of FIG. 12A, the same coil of wire (i.e., transduction coil 1225) can be used by the magnetostrictive transducer for inducing ultrasound waves 1231 and measuring reflected ultrasound waves 1232. In alternative implementations, the magnetostrictive transducer can employ two separate coils of wire-one coil for inducing ultrasound waves 1231 and another coil for measuring reflected ultrasound waves 1232. To this end, FIG. 12C illustrates one such example implementation of a magnetostrictive transducer that includes a transmission coil 1226 and a receiver coil 1227. In this example, the two separate coils can operate in a “pitch-catch” mode.

In some implementations, at least the portion(s) of the wire waveguide 1211 that are magnetized can include a material that increases a magnetostrictive coefficient of the portion(s) relative to a base material of the wire waveguide 1211, thereby creating stronger ultrasound signals in the waveguide. To this end, FIG. 12D illustrates one example implementation of a wire waveguide 1212 that includes a portion with a coating 1215 that increases the magnetostrictive coefficient. The coating 1215 can be a metal or metal alloy coating having a relatively high magnetostrictive coefficient. For example, an electroplated nickel layer can be added in particular implementations.

Another application for multiple coils of a magnetostrictive transducer along a wire waveguide is shown in FIG. 12E, which illustrates an implementation that includes multiple transduction coils 1225 coupled to a wire waveguide 1211. In this implementation, each transduction coil 1225 can be used by the magnetostrictive transducer for inducing ultrasound waves and measuring reflected ultrasound waves along the wire waveguide 1211. For example, each transduction coil 1225 can be configured to generate and measure ultrasound waves along a respective segment of the wire waveguide 1211. As such, contact location and/or force data could be determined for each respective segment based on a corresponding transduction coil. This can be used to improve the UFS measurements, particularly for wire waveguides having longer lengths and/or more contact locations. Different transduction coils 1225 could be configured to perform UFS measurements along adjacent segments of the wire waveguide 1211. As contrasted with implementations where a single transducer receiver is used, having multiple transducer receivers could help reduce the difficulty in extracting useful data from an ultrasound signal reflected from a contact location as the signal could be convolved with fewer reflected signals or not convolved at all.

In this document, the terms “machine readable medium,” “computer readable medium,” and similar terms are used to generally refer to non-transitory mediums, volatile or non-volatile, that store data and/or instructions that cause a machine to operate in a specific fashion. Common forms of machine readable media include, for example, a hard disk, solid state drive, magnetic tape, or any other magnetic data storage medium, an optical disc or any other optical data storage medium, any physical medium with patterns of holes, a RAM, a PROM, EPROM, a FLASH-EPROM, NVRAM, any other memory chip or cartridge, and networked versions of the same.

These and other various forms of computer readable media may be involved in carrying one or more sequences of one or more instructions to a processing device for execution. Such instructions embodied on the medium, are generally referred to as “instructions” or “code.” Instructions may be grouped in the form of computer programs or other groupings. When executed, such instructions may enable a processing device to perform features or functions of the present application as discussed herein.

In this document, a “processing device” may be implemented as a single processor that performs processing operations or a combination of specialized and/or general-purpose processors that perform processing operations. A processing device may include a CPU, GPU, NPU, APU, DSP, FPGA, ASIC, SOC, and/or other processing circuitry.

The terms “substantially” and “about” used throughout this disclosure, including the claims, are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

To the extent applicable, the terms “first,” “second,” “third,” etc. herein are merely employed to show the respective objects described by these terms as separate entities and are not meant to connote a sense of chronological order, unless stated explicitly otherwise herein.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present disclosure. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

It should be appreciated that all combinations of the foregoing concepts (provided such concepts are not mutually inconsistent) are contemplated as being part of the inventive subject matter disclosed herein. In particular, all combinations of claimed subject matter appearing in this disclosure are contemplated as being part of the inventive subject matter disclosed herein.

What is claimed is:

1. A system, comprising:
an ultrasound waveguide;
an ultrasound transducer assembly configured to acoustically couple to the ultrasound waveguide, the ultrasound transducer assembly configured to:
generate an ultrasound wave signal that travels along a major axis of the ultrasound waveguide and produces one or more partial reflections associated with one or more contact locations of the ultrasound waveguide having an altered acoustic impedance due to an externally applied contact force at the one or more contact locations;
receive a reflected ultrasound waveform signal including data representative of the one or more partial reflections; and
a processing device configured to estimate, based on the reflected ultrasound waveform signal, each of the one or more contact locations.

2. The system of claim 1, wherein estimating, based on the reflected ultrasound waveform signal, each of the one or more contact locations comprises determining, based on an ultrasound wave speed inside the ultrasound waveguide, and a measured time from transmission of the ultrasound wave signal to reception of the reflected ultrasound waveform signal, each of the one or more contact locations.

3. The system of claim 1, wherein the processing device is further configured to estimate, using at least amplitude information of the reflected ultrasound waveform signal, the externally applied contact force at the one or more contact locations.

4. The system of claim 3, wherein:
the one or more contact locations include a plurality of contact locations; and
estimating the externally applied contact force at the one or more contact locations comprises estimating a force applied at each of the plurality of contact locations.

5. The system of claim 3, wherein:
the one or more contact locations include a plurality of contact locations; and
estimating the externally applied contact force at the one or more contact locations comprises estimating a continuous force profile across a region of the ultrasound waveguide that includes the plurality of contact locations.

6. The system of claim 3, wherein estimating, using at least the amplitude information of the reflected ultrasound waveform signal, the externally applied contact force at the one or more contact locations comprises mapping, based on force versus amplitude calibration data associated with the ultrasound waveguide, the amplitude information to force information.

7. The system of claim 3, wherein:
the reflected ultrasound waveform signal comprises a time-domain voltage signal; and
estimating the one or more contact locations and the externally applied contact force comprises mapping the time-domain voltage signal to an output indicative of force as a function of position along the ultrasound waveguide.

8. The system of claim 1, further comprising a sheath encapsulating the ultrasound waveguide, the sheath configured to transmit the externally applied contact force to the ultrasound waveguide at the one or more contact locations.

9. The system of claim 8, wherein an inner surface of the sheath comprises one or more indenters oriented toward the ultrasound waveguide, each of the one or more indenters configured to contact a respective one of the one or more contact locations responsive to the externally applied contact force.

10. The system of claim 9, wherein each of the one or more indenters is oriented to transfer the externally applied contact force to the ultrasound waveguide along a single axis.

11. The system of claim 1, further comprising a component having a contact surface mechanically coupled to the ultrasound waveguide or a sheath encapsulating the ultrasound waveguide, the component configured to transfer the externally applied contact force from the contact surface to the ultrasound waveguide or the sheath, and the processing device configured to sense contact at the contact surface by estimating, based on the reflected ultrasound waveform signal, each of the one or more contact locations.

12. The system of claim 11, wherein the component is a gripping device configured to generate the externally applied contact force at the contact surface.

13. The system of claim 11, wherein the component is a medical device or a robotic device.

14. The system of claim 1, wherein prior to estimating each of the one or more contact locations, the processing device is further configured to remove, using a reference signal obtained when no external contact force is applied to the ultrasound waveguide, noise from the reflected ultrasound waveform signal.

15. The system of claim 1, wherein the ultrasound waveguide has a circular or rectangular cross section.

16. The system of claim 15, wherein the ultrasound waveguide is a metallic sheet waveguide having a rectangular cross section.

17. The system of claim 15, wherein the ultrasound waveguide is a wire waveguide having a circular cross section.

18. The system of claim 17, wherein the ultrasound transducer assembly comprises
a piezoelectric transducer; or
a magnetostrictive transducer including a first coil of wire configured to be placed around a first portion of the wire waveguide, and the magnetostrictive transducer is configured to generate the ultrasound wave signal by passing a time-varying current through the first coil of wire placed around the first portion of the wire waveguide in the presence of a bias magnetic field.

19. The system of claim 18,
wherein the magnetostrictive transducer is configured to receive the reflected ultrasound waveform signal via the first coil of wire; or
the magnetostrictive transducer further comprises a second coil of wire configured to be placed around a second portion of the wire waveguide, and the magnetostrictive transducer is configured to receive the reflected ultrasound waveform signal via the second coil of wire.

20. A method, comprising:
generating, by an ultrasound transducer assembly acoustically coupled to an ultrasound waveguide, an ultrasound wave signal that travels along a major axis of the ultrasound waveguide and produces one or more partial reflections associated with one or more contact locations of the ultrasound waveguide having an altered acoustic impedance due to an externally applied contact force at the one or more contact locations;
receiving, by the ultrasound transducer assembly, a reflected ultrasound waveform signal including data representative of the one or more partial reflections; and
estimating, by a processing device, based on the reflected ultrasound waveform signal, each of the one or more contact locations.

21. The method of claim 20, further comprising applying the externally applied contact force at the one or more contact locations.

22. The method of claim 21, wherein applying the externally applied contact force comprises applying the externally applied contact force via a contact surface of a device coupled to the ultrasound waveguide.

23. The method of claim 22, wherein the device is a gripping device, the contact surface is a gripping surface of the gripping device, and applying the externally applied contact force comprises gripping an object with the gripping device.

24. The method of claim 20, further comprising estimating, by the processing device, using at least amplitude information of the reflected ultrasound waveform signal, the externally applied contact force at the one or more contact locations.

25. The method of claim 23, wherein:
the reflected ultrasound waveform signal comprises a time-domain voltage signal; and
estimating the one or more contact locations and the externally applied contact force comprises mapping the time-domain voltage signal to an output indicative of force as a function of position along the ultrasound waveguide.
